(19) United States
(12) Patent Application Publication
(10) Pub. No.: US 2010/0143370 A1
(43) Pub. Date: Jun. 10, 2010

(54) SET OF MEANS FOR TREATING A MALIGNANT PATHOLOGY, AN AUTOIMMUNE DISEASE OR AN INFECTIOUS DISEASE

(75) Inventors: Christophe De Romeuf, Lambersart (FR); Nathalie Fournier, Marcq En Baroeul (FR); Nadine Fernandez, Issy-Les-Moulineaux (FR)

Correspondence Address: HARNESS, DICKEY & PIERCE, P.L.C., P.O. BOX 828, BLOOMFIELD HILLS, MI, 48303, US

(73) Assignee: LFB BIOTECHNOLOGIES, Les Ulis (FR)

(21) Appl. No.: 12/597,471

(22) PCT Filed: Apr. 25, 2008
(86) PCT No.: PCT/FR08/00598

§ 371 Date: Oct. 23, 2009

(30) Foreign Application Priority Data
Apr. 25, 2007 (FR) 0703013

(51) Int. Cl. A61K 39/395; A61P 37/04; A61P 35/00; A61P 31/04; A61P 31/12

(52) U.S. Cl. 424/145.1; 424/141.1; 424/156.1

(57) ABSTRACT

Kit of parts for treating a malignant pathology, an auto-immune disease or an infectious disease, comprising an effector cell which expresses the FcγRIII receptor (CD16) on its surface, and a monoclonal antibody, the affinity of the Fc region of said monoclonal antibody for CD16 being greater than the affinity of the Fc region of the polyclonal immunoglobulins for CD16.

### CROSS-REFERENCE TO RELATED APPLICATIONS

This application is a National Phase Entry of International Application No. PCT/FR2008/000598, filed on Apr. 25, 2008, which claims priority to French Patent Application 07 03 013, filed on Apr. 25, 2007, both of which are incorporated by reference herein.

### BACKGROUND

The present invention relates in general to treating a malignant pathology, an auto-immune disease, or an infectious disease, especially by means of an effector cell which expresses an FcγR receptor on its surface.

Used more and more in research, antibodies are also tools of choice in diagnostics and therapeutics, where they represent an alternative to conventional treatments. Numerous preparations of antibodies for therapeutic usage, of plasmatic origin or originating from biotechnologies, are currently on the market, or in clinical development phase. Their properties are exploited to produce therapeutic tools capable of binding specifically with their target, and efficiently recruiting the cells of the immune system.

Research has recently focussed on improving the efficiency of antibodies, and more particularly on manipulation of their constant Fc region. It is the latter which is responsible for the “effector” properties of antibodies, since it allows mobilisation of the effector cells of the immune system and of proteins of the complement. This faculty is made possible by the presence, on certain effector cells, of glycoproteins i.e. the receptors Fc (FcR). These receptors are capable of binding the constant region of the antibodies, once the latter are fixed, by their variable region, on the target antigen. The binding of the Fc region of antibodies on the FcR carried by the effector cells causes in the latter activation of cytotoxic mechanisms such as phagocytosis and ADCC (cellular cytotoxicity dependent on antibodies or Antibody-Dependent Cell-mediated Cytotoxicity). During an auto-immune disease, the immune system, the natural role of which is to protect the organism from aggression, causes an inflammatory response in the absence of extraneous bodies and thus itself causes tissue lesions by “accidentally” attacking the molecules of the self. There are different auto-immune pathologies affecting different tissues or different functions of the body. For example, the brain is affected in those people suffering from multiple sclerosis, the intestines are the target in patients afflicted with Crohn's disease and synovia, bones and cartilages are affected in those afflicted by rheumatoid polyarthritis.

During development of the auto-immune disease, several phenomena may eventuate, such as progressive destruction of one or more types of tissue, abnormal growth of an organ or modifications of the function(s) of the organ affected. The tissues or organs most often affected during an auto-immune disease are haematopoietic cells, blood vessels, connective tissues, endocrine glands, muscles, joints and skin. Auto-immune diseases are often associated with a chronic inflammatory pathology. The most frequent case is represented by rheumatoid polyarthritis and juvenile rheumatoid arthritis which are two types of inflammatory arthritis. Arthritis is a general term designating inflammation of joints.

The plurality of treatments has numerous secondary effects or do not fully prevent progression of the disease. There is currently no ideal treatment and none to help cure patients, culminating in an obvious need for novel therapies which are more efficient and above all curative. Since B lymphocytes (LB) are those cells producing auto-antibodies often responsible for the development of auto-immune diseases, their destruction by administration of a specific monoclonal antibody of this cellular type may be only beneficial to patients, as shown for rituximab recently approved for treating rheumatoid polyarthritis.

Also, despite considerable progress made in improving sanitary conditions, in immunisation and in anti-microbial therapies, infectious diseases represent a persistent and significant problem for modern medicine. The most widespread disease, the simple cold, is an infectious disease in the same category as AIDS (acquired immune deficiency syndrome), the most feared disease. It has been proven that certain neurological disorder classes such as degenerative diseases were in fact associated with infection.

Infectious diseases are set to remain a major medical problem in the future. During an infectious disease, the monoclonal antibodies may play two complementary roles: a neutralising role of the pathogenic agent or toxins secreted during the acute phase of infection and a destroying role of reservoir cells during transition to the chronic phase.

The destruction of host cells enabling low-noise duplication of the pathogenic agent could prevent passage to a chronic phase ending most often in the development of serious pathologies such as an auto-immune disease or a cancer. These days, despite the existence of a real need, there is almost no efficacious anti-infectious treatment in the treatment of chronic phases. On the other hand, the beneficial effects of small molecules (antibiotic, anti-parasitic, anti-viral) during the acute phases of infections are becoming increasingly compromised by the development of cross-resistance. Therefore the appearance of bacteria multi-resisting to antibiotics poses a problem of public health with 6% to 7% of all hospitalisations complicated by more or less serious nosocomial infection, or around 750,000 cases of the 15 millions hospitalisations annually (http://www.senat.fr/rap/r05-21/r05-4211.html#toc13). In total, nosocomial infections would thus be responsible of 9000 deaths every year, whereof 4200 concern patients for whom the vital prognosis was not engaged in the short term on their entering hospital (http://www.senat.fr/rap/r05-421/r05-4213.html). Therefore, it appears necessary to develop innovative drugs which will offer a therapeutic alternative for doctors and their patients.

A tumour corresponds to a neoplasic mass resulting from uncontrolled proliferation of cells which may be benign or malignant. Benign tumours generally remain localised. Malignant tumours are collectively called cancers. The term “malignant” in general means that the tumour is capable of invading and destroying adjacent structures and diffusing towards remote sites, in the long run causing the death of the patient (Robbins and Angell, 1976, Basic Pathology, 2d Ed., W.B. Saunders Co., Philadelphia, pp. 68-122). Cancer may arise in numerous different locations and behave differently as a function of its tissular origin. Currently, means for treating cancers are surgery, chemotherapy, hormonal therapy and/or irradiation for eradicating tumour cells present in the patient (Stockdale, 1998, “Principles of Cancer Patient Management”, in Scientific American Medicine, vol. 3, Rubenstein and Federman, Eds., Chapter 12). All these treatments have major drawbacks. For example, despite the availability of a large variety of chemical molecules, chemotherapy causes numerous side-effects, such as severe nausea, medullar aplasia, immunosuppression, etc. The gravity of some of these effects obliges the doctor to sometimes discontinue treatment. In addition, despite administration of a combination of several chemical molecules, most of tumour cells is resisting or develop resistance to chemotherapy agents. In fact, cells resisting to a particular agent administered in the current protocol, are unfortunately also resisting to other drugs, including those acting via a mechanism different to that used by the agent administered in the treatment protocol. This phenomenon, known under the name of multidrug resistance, is often the origin of the therapeutic failure of standard chemotherapy protocols. There is thus a real need for innovative anticancer therapies, in particular for treating cancers refractory to conventional treatment such as surgery, irradiation, chemotherapy or hormonal therapy.

A promising alternative is immunotherapy, in which the tumour cells are specifically targeted by the antibodies which are specific for tumorous antigens. Major efforts have been made to exploit the specificity of the immune response, for example hybridome technology has enabled the development of monoclonal antibodies which are specific for antigens expressed by tumour cells (Green M. C. et al., 2000 Cancer Treat Rev., 26: 269-286; Weiner L. M., 1999 Semin Oncol. 26 (suppl. 14):43-51). The destruction of harmful cells of the host or pathogenic agents corresponds to the desired efficiency mechanism of monoclonal antibodies irrespective of the targeted pathology. It is thus critical for these antibodies to be improved so as to interact optimally with the effector cells of the immune system of the patient.

Chronic lymphoid leukaemia B (LLC-B), a disease characterised by malignant proliferation of B lymphocytes (LB), is the most frequent form of leukaemia. Current treatment is essentially based on therapeutic abstinence for the early stages of the disease. In the event of clinical or haematological symptomatology, patients are classically treated by corticoids alone or in association with anti-mitotic molecules. For most of patients, resistance to treatment sets in more or less long term and generally ends in the failure of the therapeutic effort with the appearance of chemo-resisting cells. Chemotherapy is responsible for substantial side effects, especially with myelotoxicity generating an immune deficit responsible for the appearance of serious infections, sometimes deadly, in patients. Many therapeutic approaches focussed on destroying tumorous B cells as specifically as possible have been evaluated. The specific expression of the CD20 molecule by the tumorous (and normal) LB has allowed development of therapies based on the use of human anti-CD20 monoclonal antibodies.

A single non-radio labelled anti-CD20 monoclonal antibody, rituximab (Rituxan®, Genentech and Mabthéra™, Roche), is currently available commercially. It is indicated for treating patients affected by follicular lymphomas of stage III-IV and in association with chemotherapy for treating patients presenting diffuse CD20 positive large B cells aggressive non-Hodgkin's lymphoma (NHL). Since its efficiency remains variable and often modest when used as single agent (Teeling et al. 2004, Blood 104 (6)1793-800), it is the most often used in association with chemotherapy.

Rituximab has also been evaluated in patients with LLC-B. This antibody having presented only slight efficiency when used in monotherapy, it is currently administered in association with chemotherapy. Many reasons may explain the failure of monotherapy by rituximab in patients affected by LLC-B: first, rituximab in vitro causes slight ADCC activity on B cells, and, contrary to normal LB and in NHL, LB of LLC-B express only few CD20 molecules on their surface (density about 5 times less, quantitative measure by flow cytometry), thus limiting the quantity of antibodies on the cellular surface and thus the associated cytotoxic functions (ADCC and activity complement especially). It is thus of major importance to focus on alternative therapies including antibodies which are specific for the CD20 antigen and capable of efficiently causing lysis in tumour cells, including those slightly expressing the antigen.

Macrophages, effector cells of inherent immunity, play a major role in anti-tumorous responses. Naturally present in an inactive form (in the absence of any pathology), they may be activacted in vivo or in vitro by different routes, such as ingestion of pathogens or binding to receptors expressed at the surface of immune complexes (binding to FcR via the Fc region of antibodies) or cytokines, immuno-modulatory molecules produced especially during an inflammatory phenomenon. Activation induces lytic and thus increased anti-tumorous activity in macrophages (Adams D. and Hamilton T.: Activation of macrophages for tumour cell kill: effector mechanism and regulation. In Heppner & Fulton (Eds), Macrophages and cancer. CRC Press, 1988, p. 27; Fidler I.: Macrophages and metastases. A biological approach to cancer therapy. Cancer Res, 45: 4714, 1985).

In addition, macrophages, or other cells derived from monocytes or their precursors, are antigens presenting cells. Due to their strong capacity for endocytosis, digestion and presentation to T lymphocytes of antigenic peptides associated with molecules of the of major histocompatibility complex (MHC), they are capable of inducing a specific immune response.

With the aim of increasing the efficiency of rituximab, its association with macrophages activated ex vivo in the presence of interferon-γ (IFN-γ) was evaluated in vitro in a test for lysis of primary cells of LLC-B (Lefebvre M L, Stefan W. Krause, Salcedo M, Nardin A. Ex vivo activated human macrophages kill chronic lymphocytic leukemia cells in the presence of Rituximab: mechanism of antibody-dependent cellular cytotoxicity and impact of human serum. J. Immunother; vol. 29, no. 4: 388-397, 2006). The results indicate that the strong lysis of LLC-B cells by activated macrophages in the presence of rituximab is strongly inhibited by increasing concentrations of human serum. This inhibition is linked to competition by polyclonal immunoglobulins which are present in the serum vis-à-vis the binding of the rituximab-LLC-cell complex to the various FcR expressed at the surface of the macrophages. The intensity of this inhibition depends on the used concentrations of rituximab and effector cells: target cells ratio (Effector:Target or E:T). Despite the existence of numerous therapeutic tools for treating cancers, auto-immune diseases, or infectious diseases, there is still a substantial need for novel immunotherapy products, evidencing greater efficiency and greater safety than existing products.

### SUMMARY

A primary object of the invention is a kit of parts for treating a malignant pathology, an auto-immune disease or an infectious disease, comprising an effector cell which expresses the FcγRIII receptor (CD16) on its surface, and a monoclonal antibody, in which the affinity of the Fc region of said monoclonal antibody for CD16 is greater than the affinity of the Fc region of the polyclonal immunoglobulins for CD16. Advantageously, the effector cell which expresses the FcγRIII receptor (CD16) on its surface is a monocyte or a cell derived from a monocyte or a monocyte precursor which expresses the FcγRIII receptor (CD16) on its surface. Advantageously, this cell is selected from monocytes expressing CD16, macrophages, Natural Killer cells (NK), dendritic cells and peripheral blood mononuclear cells as a whole (Peripheral Blood Mononuclear Cell or PBMC). Advantageously, the cell expressing CD16 on its surface is selected from monocytes expressing CD16, macrophages and dendritic cells.

More particularly, the monocyte derived cell a monocyte precursor, which expresses CD16 on its surface, is a macrophage. Advantageously, the monoclonal antibody is not displaced by polyclonal immunoglobulins, particularly those present in the serum, due to the high affinity of the Fc region of said monoclonal antibody for CD16. Advantageously, the monoclonal antibody binds to CD16 of said monocyte or monocyte precursor derived cell with an affinity greater than 2.10⁶M⁻¹.

In a particularly advantageous manner, the monoclonal antibody is produced in the form of a monoclonal antibodies composition, in which each antibody has sugar chains bound to N at the Fcγ glycosylation site (asparagine 297, according to Kabat), and in which, among all sugar chains which are bound to N at said glycosylation site of all the antibodies of said composition, the fucose rate is less than 65%. In a particular embodiment of the invention, the monoclonal antibody is directed against an antigen selected from antigen 5C5 (tumorous antigen expressed by the cells of renal carcinomas), BCR (B Cell Receptor), an idiotype such as that of anti-FVIII inhibitor antibodies, TCR (T Cell Receptor), CD2, CD3, CD4, CD8, CD14, CD15 CD19, CD20, CD21, CD22, CD23, CD25, CD45, CD30, CD33, CD37, CD38, CD40, CD40L, CD46, CD52, CD54, CD66 (a, b, c, d), CD74, CD80, CD86, CD126, CD138, CD154, MUC1 (Mucine 1), MUC2 (Mucine 2), MUC3 (Mucine 3), MUC4 (Mucine 4), MUC16 (Mucine 16), HM1.24 (specific antigen for plasmocytes which is overexpressed in multiple myelomas), tenascin (protein of the extra-cellular matrix), GGT (gamma-glutamyltranspeptidase), VEGF (Vascular Endothelial Growth Factor), EGFR (Endothelial Growth Factor receptor), CEA (carcinoembryonic antigen), CSAp (colon-specific antigen-p), ILGF (Insulin-Like Growth factor), placental growth factor, Her2/neu, carbonic anhydrase IX, IL-6, proteins S100 (multigenic family of proteins binding to calcium), MART-1 (tumorous differentiation antigen associated with melanoma), TRP-1 (tyrosinase-related protein 1), TRP-2 (tyrosinase-related protein 2), gp100 (glycoprotein 100 kDa), amyloid proteins, rhesus D antigen, MHC molecules of class I and II such as HLA-DR), an antigen resulting from the expression of mutated genes, especially oncogenes or tumour-suppressor genes, an antigen derived from oncogene virus which are expressed by certain well defined tumours, a ubiquitous antigen overexpressed in some tumours and slightly expressed in some normal tissues, such as for example the type II receptor of the Müllerian hormone, a glycosylated or non-glycosylated protein, a phospholipid, a molecule of the self or of the non self expressed or exposed on the membrane by infected cells such as phosphatidylserine, and a protein expressed or secreted by a pathogenic agent (bacterial toxin, protein complexes of the bacterial or parasitic wall, viral envelope glycoproteins, for example from HIV virus, HBV, HCV and RSV, etc.).

Preferably, the monoclonal antibody is directed against CD20. In a preferred embodiment of the invention, the anti-CD20 antibody is produced by the cell line R509 deposited to the CNCM on Nov. 8, 2004 under the accession number I-3314, or by the cell line R603, deposited to the CNCM on Nov. 29, 2005 under accession number I-3529 (CNCM: Collection Nationale de Culture de Microorganismes, Institut Pasteur, 25 rue du Docteur Roux, 75724 Paris Cedex 15-France). Advantageously, the kit of parts of the invention is intended for use in therapy, simultaneously, sequentially or separately. Advantageously, in the kit of parts of the invention, the effector cell expressing CD16 on its surface has a cytotoxic activity on the cell targeted by said antibody, which is favoured by the interaction of the antibody with CD16. Advantageously, in the kit of parts of the invention, the monoclonal antibody induces cytotoxicity by ADCC activity or by phagocytosis of said antibody targeted cell in the presence of an effector cell expressing CD16.

Another object of the invention is a pharmaceutical composition containing the kit of parts according to the invention, and pharmaceutically acceptable excipients. Another object of the invention relates to the use of the kit of parts of the invention for manufacturing a drug.

Another object of the invention relates to the use of the kit of parts of the invention for manufacturing a drug intended for treatment of a malignant pathology.

Advantageously, the malignant pathology is selected from solid tumours and malignant haemopathies. Advantageously, the solid tumours are selected from melanomas, carcinomas, sarcomas, gliomas and skin cancers. Advantageously, the carcinomas are selected in the group consisting of kidney, breast, oral cavity, lungs, gastro-intestinal tract, ovaries, prostate, uterus, bladder, pancreas, liver, gallbladder, skin and testicles carninomas. Advantageously, malignant haemopathies are selected from lymphoproliferative, myeloproliferative, myelodysplasic syndromes and acute myeloid leukemias with, for example, type B NHL, acute or chronic B lymphoid leukemias, Burkitt's lymphoma, tricholeucocyte leukaemia, acute and chronic myeloid leukemias, T lymphomas and leukemias, Hodgkin's lymphomas, Waldenström's macroglobulinemia and multiple myelomas.

Another object of the invention relates to the use of the kit of parts of the invention for manufacturing a drug for the treatment of an auto-immune and/or of a primitive or secondary inflammatory disease, which is specific to organs or systemic and which is associated or not with pathogenic auto-antibodies. Advantageously, the auto-immune and/or inflammatory disease is selected from organ graft rejection, or graft versus host disease, rheumatoid polyarthritis, disseminated lupus erythematosus, sclerodermia, primitive Sjögren's syndrome (or Gougerot-Sjögren syndrome), auto-immune polyneuropathies such as multiple sclerosis, type I diabetes, auto-immune hepatitis, ankylosing spondylarthritis, Reiter's syndrome, gout arthritis, coeliac disease, Crohn's disease, Hashimoto's thyroiditis, Addison's disease, auto-immune hepatitis, Basedow's disease, ulcerative colitis, vasculitis such as systemic vasculitis associated with ANCA (antineutrophil cytoplasmic antibody), auto-immune cytopenias and other haematological complications in adults and children, such as acute or chronic auto-immune thrombopenias, auto-immune haemolytic anaemias, haemolytic disease of the newborn (HDN), cold agglutinin disease, thrombocytopenic thrombotic purpura and acquired auto-immune haemophilia; Goodpasture's syndrome, extra-membraneous nephropathies, auto-immune bullous skin disorders, refractory myasthenia, mixed cryoglobulinemias, psoriasis, juvenile chronic arthritis, inflammatory myositis, dermatomyositis and children systemic autoimmune diseases including the antiphospholipide syndrome.

Another object of the invention relates to the use of the kit of parts of the invention for manufacturing a drug for the treatment of an infectious disease. Advantageously, the infectious disease is selected from those induced by viruses (human immunodeficiency virus or HIV, hepatitis B or C virus (HBV, HCV), Epstein-Barr virus or EBV, cytomegalovirus or CMV, enterovirus, influenza with Influenza virus A, B and C, syncytial respiratory virus or SRV, or HTLV), bacteria and/or their toxins (tetanus, diphtheria, pneumococci, meningococci, staphylococci including methicilin resistant forms, Klebsiellas, Shigellas, pseudomonas aeruginosa, enterobacteria or antiotic resisting pathologies including nosocomial diseases), parasites (paludism, leishmaniosis, trypanosomiasis) as well as emerging diseases, for example Chikungunya, bird flu, severe acute respiratory syndrome virus or SARS, viruses responsible for haemorrhagic fevers such as Ebola or Dengue fever or West Nile virus, and those related to bio-terrorism, such as Anthrax, Botulism, Plague, smallpox and poxvirus, Tularaemia, haemorrhagic fever agents, brucellosis, Staphylococcus B Enterotoxins, diphtheric toxin or viral Encephalitis.

### DETAILED DESCRIPTION

### Kit of Parts

The term “kit of parts” designates a drug combination the elementary constituents of which form a functional unit due to their common indication. More specifically, the kit of parts of the invention is a drug combination containing, as active substance, an effector cell which expresses CD16 on its surface and a monoclonal antibody in which the affinity of the monoclonal antibody Fc region for CD16 is greater than the affinity of the polyclonal immunoglobulins Fc region for CD16, for simultaneous, separate or sequential use, for the treatment of malignant pathologies, auto-immune or infectious diseases.

The monoclonal antibody and the effector cell, which expresses CD16 on its surface, together form a composition in the form of an unitary kit of parts, the constituents of which are available for simultaneous, separate or staggered over time application. The kit of parts of the invention may also be in the form of a mixture. The monoclonal antibody and the effector cell which expresses CD16 form a functional unit due to a novel common effect and thus a common indication.

Effector Cell which Expressing the FcγRIII Receptor (CD16) on its Surface

By “Effector cell expressing the CD16 receptor on its surface”, it is meant any cell capable of an effector activity (in particular cytotoxic activity by ADCC, phagocytosis or, in another field, of antigenic presentation and humoral response properties) following cellular activation induced by the binding of an immune complex formed by the association of an antibody with the antigen it is specific for the FcγRIII or CD16 membraneous receptor. These cells necessarily express CD16 on their surface. Advantageously, such a cell may be a cell derived from monocyte or a monocyte precursor derived-cell which expresses CD16 on its surface, a monocyte CD16+, a macrophage, a dendritic cell, this list not being limited.

Therefore, this list may also extend to Natural Killer (NK) and PBMC (Peripheral Blood Mononuclear Cell) cells. By “NK cells” it is meant large granulosar lymphocytes capable of a spontaneous cytotoxic activity without previous immunisation. By “PBMC” it is meant any mononucleated cell of the peripheral blood (monocytes and lymphocytes), and which expresses CD16 on its surface. Such cells are thus capable of inducing ADCC activity in the presence of the monoclonal antibodies of the invention, due to the binding between the monoclonal antibodies Fc region and the CD16 receptor expressed by the cell. The effector cell is preferably a macrophage. The CD16+ monocyte (i.e. expressing CD16 on its surface) is a monocyte sub-population expressing CD16 on its membrane surface. CD16+ monocytes are capable of phagocyting and inducing ADCC activity.

The macrophage is one of the main players of inherent immunity and participates to the adaptive immunity. It comes from the differentiation of monocytes. By way of example, macrophages may be derived from a cellular suspension strongly enriched in monocytes comprising a culture step in a suitable culture medium (RPMI® medium for Roswell Park Memorial Institute) containing M-CSF (Monocyte-Colony Stimulating Factor) or GM-CSF (Granulocyte Macrophage-Colony Stimulating Factor) to induce differentiation of monocytes into macrophages. The latter may be generated, for example, in six to seven days of culture.

It is also possible to produce macrophages from a composition enriched with blood cells obtained by cytapheresis carried out on a healthy individual, and by conducting a step for culturing monocytes in a culture medium containing M-CSF (Monocyte Colony Stimulating Factor) or GM-CSF (Granulocytes Macrophages Colony Stimulating Factor). Optionally, this culture step may advantageously be preceded firstly by a separation step of, firstly, mononuclear cells, and, in an other hand, red blood cells, granulocytes and part of the platelets contained in the blood derived composition obtained by cytapheresis, and by an elimination step, by washing of a part of the blood platelets and anticoagulants remaining than the preceding step. The abovementioned enriching step of the cellular suspension in monocytes is generally achieved by centrifugating the medium containing the monocytes on a density gradient, especially on a solution having a density of about 1.0 to about 1.3 g/ml, such as a solution of Ficoll Paque type (Pharmacia) having a density of 1.077 g/ml.

Optionally, a composition containing macrophages, and/or dendritic cells, and/or NK cells may be obtained starting from a blood derived composition of human origin, and enriched in blood cells, and, more particularly, in white blood cells such as monocytes, or precursors thereof, especially a blood derived composition such as those obtained by cytapheresis, said process comprising the following steps:

Advantageously, diluting of said blood derived composition, especially in about 2 to 3 times the volume thereof, by means of a suitable physiological solution,

washing said blood derived composition, advantageously by simple centrifugation and washing of the pellet resulting from the above-mentioned centrifugation, after recovery of the pellet, in suitable physiological washing solution, especially in a pocket (of transfer pocket type), by exerting pressure for example on said pocket, the washing solution then being eliminated to another pocket or other receptacle, to recover a composition deprived of any possible anticoagulants and of any diverse residues, and impoverished in platelets,

if required, repeating the abovementioned washing step, especially between 1 and 2 times,

culturing the cells contained in the blood derived composition obtained after the abovementioned washing step, by placing the latter in an appropriate culture medium, especially in an advantageously hydrophobic pocket, for about 6 to about 10 days (especially about 6 to 7 days),

this culture step being:

preceded by an elimination step of all or part of the constituents other than the monocytes, or their precursors, which are likely to be present in the starting composition, especially platelets, red blood cells, granulocytes and lymphocytes, by placing the blood derived composition obtained after the washing step preceding the culture step in contact with antibodies directed against all or part of the abovementioned constituents, and recovering the solution containing the monocytes, or their precursors, while all or part of the abovementioned constituents remain fixed to the antibodies, and/or followed by an elimination step of all or part of the constituents other than the macrophages by contacting the blood derived composition obtained after the culture step with the antibodies such as described hereinabove, and recovering the solution containing the macrophages, while all or part of the abovementioned constituents remain fixed to the antibodies,

and/or followed by a purification step, especially by elutriation, in which the macrophages are physically separated from the other constituents of the composition obtained after the culture step, especially from the platelets, red blood cells and lymphocytes.

More generally, any other process for obtaining macrophages, resulting in the expression of CD16 on their surface, is also applicable to the invention. In addition, by “macrophage”, in the present invention, it is meant any cell obtained from monocytes and which is differentiated according to a well determined protocol, thus resulting in cells expressing the following membrane markers: CD14+, CD16+, CD32+, CD64+, CD11b+. In particular, the percentage of CD16+ cells is of at least 20%, preferably 50%, or 70% or is comprised between 70 and 100%.

Monoclonal Antibodies

For the purposes of the invention, the expressions “monoclonal antibody” or “composition of monoclonal antibody” refer to a preparation of antibody molecules originating from a cellular clone and having an identical and single specificity. A molecule of immunoglobulin is composed of 4 polypeptides: 2 identical heavy chains (H, Heavy) of 50 kDa each and 2 identical light chains (L, Light) of 25 kDa each. The light chain is composed of 2 domains, a variable domain V and a constant domain C, folded back independently of one another in space, called VL and CL. The heavy chain also includes a V domain noted as VH and 3 or 4 C domains noted as CH1 to CH4. Each domain comprises about 110 amino acids and is structured comparably. The 2 heavy chains are linked by disulfide bridges and each heavy chain is linked to a light chain by a disulfide bridge also.

The region determining the specificity of the antibody for the antigen is borne by the variable parts, while the constant parts may interact with the Fc receptors of the effector cells or with molecules such as proteins of the complement to cause different functional properties. As regards the variable regions of the heavy and light chains, it is observed that the variability in sequence is not distributed equally. In effect, the variable regions are constituted both by regions only slightly variable known as “framework” (FR), numbering 4 (FR 1 to FR4) and also by regions in which variability is extreme: these are “hypervariable” regions, or CDR (for Complementarity Determining Regions), totalling 3 (CDR1 to CDR3).

Advantageously, the antibody according to the invention is a chimeric, humanised or human antibody. The antibody according to the invention is preferably chimeric. By “Chimeric antibody”, it is meant an antibody, the variable regions of the light chains and the heavy chains of which belong to a different species from that the constant regions of the light chains and the heavy chains belong to. Therefore, the antibody according to the invention also has variable regions of murine, rat, rabbit, monkey, goat, or human origine and constant regions which belong to a species different from the species where the antibody was produced. In this respect, all the families and species of mammals are likely to be used, and in particular human being, monkey, rats and mice, swine, bovines, equines, felines, canines, for example, as well as birds. Even more preferably, the constant regions of each of the light chains and each of the heavy chains of the antibody according to the invention are human constant regions. This preferred embodiment of the invention allows to decrease the immunogenicity of the antibody in humans and thereby to improve its efficiency during its therapeutic administration in human.

In a preferred embodiment of the invention, the constant region of each of the light chains of the antibody according to the invention is of κ-type. Any allotype is suitable for achieving the invention, for example Km(1), Km(1, 2), Km(1, 2, 3) or Km(3). In another embodiment of the invention, the constant region of each of the light chains of the antibody according to the invention is of λ-type.

In a particular aspect of the invention, and especially when the constant regions of each of the light chains and of each of the heavy chains of the antibody according to the invention are human regions, the constant region of each of the heavy chains of the antibody is of γ-type. According to this variant, the constant region of each of the heavy chains of the antibody may be of γ1-type, of γ2-type, of γ3-type, these three types of constant regions having the particular feature of fixing the human complement, or even of γ4-type. The antibodies, the heavy chains of which have a γ type constant region belong to the class of IgG. The G-type immunoglobulins (IgG) are heterodimers constituted by 2 heavy chains and 2 light chains, linked to one another by disulfide bridges. Each chain is constituted, in the N-terminal position, by a variable region or domain (coded by the rearranged genes V-J for the light chain and V-D-J for the heavy chain) which is specific for the antigen against which the antibody is directed, and in the C-terminal position, of a constant region, constituted by a single CL domain for the light chain or of 3 domains (CH1, CH2 and CH3) for the heavy chain. The association of the variable domains and the CH1 and CL domains of the heavy and light chains forms the Fab fragments which are connected to the Fc region by a very flexible hinge region allowing each Fab to be fixed to its antigenic target while the Fc region, which mediates the effector properties of the antibody, remains accessible to the effector molecules such as the FcγR and the C1q receptors. The Fc region, constituted by 2 globular domains Cγ₂ and Cγ₃, is glycosylated at the level of the Cγ₂ domain with the presence, on each of the 2 chains, of a biantenna N-glycan, linked to asparagine 297. The constant region of each of the heavy chains of the antibody is preferably of γ1-type, since such an antibody shows the capacity to engender ADCC activity (Antibody-Dependent Cellular Cytotoxicity) in the greatest number of (human) individuals. In this respect, any allotype is suitable for achieving the invention, for example G1m(3), G1m (1, 2, 17), G1m(1, 17) or G1m(1,3).

The chimeric antibodies according to the invention may be constructed using standard recombinant DNA techniques, well known to those skilled in the art, and more particularly using the construction techniques of chimeric antibodies described for example in Morrison et al., Proc. Natl. Acad. Sci. U.S.A., 81: 6851-55 (1984), where DNA recombinant technology is used for replacing the constant region of a heavy chain and/or the constant region of a light chain of an antibody originating from a non-human mammal with the corresponding regions of a human immunoglobulin. Such antibodies and their preparation method have also been described in patent EP 173 494, in the document Neuberger, M. S. et al., Nature 1985 Mar. 21-27; 314(6008): 268-70., as well as in the document EP 125 023, for example. Methods for generating chimeric antibodies are widely available for those skilled in the art. For example, the heavy and light chains of the antibodies may be expressed separately using a vector for each chain, or they may be integrated into a single vector.

An expression vector is a nucleic acid molecule in which the nucleic acid sequence coding for the variable domain of each of the heavy or light chains of the antibody and/or the nucleic acid sequence, preferably human, coding for the constant region of each of the heavy or light chains of the antibody have been inserted, so as to introduce and keep them in a host cell. It allows expression of these foreign nucleic acid fragments in the host cell since it has indispendable sequences (promoter, polyadenylation sequence) to this expression. The vector may be for example a plasmid, an adenovirus, a retrovirus or a bacteriophage, and the host cell may be any mammalian cell, for example SP2/0, YB2/0, IR983F, Namalwa human myeloma, PERC6, CHO lines, especially CHO-K-1, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag 14 and P3X63Ag8.653.

For constructing expression vectors for the chimeric antibodies according to the invention, synthetic signal sequences and suitable restriction sites may be fused to the variable regions during PCR amplification reactions (Polymerase Chain Reaction). The variable regions are then combined with the constant regions of an antibody, preferably a human IgG1. The genes thus constructed are cloned under the control of a promoter (for example the RSV promoter) and upstream of a polyadenylation site, using either two separate vectors (one for each chain) or a single vector. The vector(s) is (are) also provided with selection genes known to those skilled in the art, such as for example the dhfr gene, the neomycin resistance gene. The chimeric antibodies according to the invention may be produced by co-transfecting or single transfecting the light chain expression vector of the heavy chain expression vector or the single vector in a host cell through the use of a method well known to those skilled in the art (for example co-precipitation with calcium phosphate, electroporation, micro-injection, etc.).

By Humanised antibody, it is meant to refer to an antibody containing CDRs regions derived from a non-human antibody, the other parts of the antibody molecule being derived from one (or more) human antibodies. Such antibodies may be prepared according to CDR grafting methods (“CDR-grafting”) well known to those skilled in the art (U.S. Pat. No. 5,225,539, U.S. Pat. No. 6,180,370; Jones et al., Nature 321(6069): 522-5. (1986); Verhoeyen et al., Bioessays 8(2): 74-8 (1988); Riechmann et al., Nature 332: 323-7 (1988); Queen C. et al. Proc. Natl. Acad. Sci. U.S.A. 86(24):10029-33 (1989); Lewis A. P. and Crowe J. S., Gene 101(2): 297-302 (1991); Daugherty B L et al. Nucleic Acids Res. 19(9): 2471-6 (1991); Carter et al. Proc. Natl. Acad. Sci. USA, 89: 4285 (1992); Singer et al., J. Immunol. 150 (7): 2844-57 (1993); Presta et al., J. Immunol., 151: 2623 (1993)). The choice of variable human domains to be grafted for producing humanised antibodies is important for reducing the antibody immunogenicity without altering its affinity for its target. In a production method for a humanised antibody, the sequence of the variable domain of a murine antibody is compared to a sequence library of known variable human regions and the closest variable human sequence to the murine sequence is retained as FR region (“framework”) of the humanised antibody [Riechmann et al., Nature 332: 323-7 (1988); Queen C. et al., Proc. Natl. Acad. Sci. USA 86(24): 10029-33 (1989); Sims et al., J. Immunol., 151: 2296 (1993)]. Another selection method of human FR regions is the comparing the sequence of each murine FR sequence sub-region (FR1, FR2, FR3 and FR4) with a known human FR sequences library, such as to select, for each FR region, the closest human FR sequence to the murine sequence [US patent 2003/0040606; Singer et al., J. Immunol. 150 (7): 2844-57 (1993); Sato K. and et al, Mol. Immunol. 31(5): 371-81 (1994); Leung S. O. et al., Mol. Immunol. 32 (17-18): 1413-27 (1995)]. Another method uses a particular FR region which is derived from a consensus sequence of a particular sub-group of human antibodies heavy or light chain [Sato K. and et al, Mol. Immunol. 31(5): 371-81 (1994)]. The CDR graft is completed in the majority of cases by muting some key residues localised in human FRs in order to conserve a good affinity of the humanised antibody for its target [Holmes M. A. and Foote J., J. Immunol. 158(5): 2192-201 (1997)].

The humanised antibodies according to the invention are preferred for their use in in vitro diagnostic methods, or in vivo prophylactic and/or therapeutic treatment methods. The thus chimerised or humanised antibody according to the invention has the advantage of being better tolerated by the human organism, and at least as efficient as the original antibody. In a particularly advantageous way, the thus chimerised or humanised antibody is twice as cytotoxic as the corresponding native antibody. In an even more advantageous way, the thus chimerised or humanised antibody is 10 times, or even 100 times or preferably more than 500 times more cytotoxic than the corresponding native antibody.

By human antibody, it is meant to refer to an antibody each region of which is derived from a human antibody. These antibodies may be derived from transgenic mice carrying human antibodies genes or from human cells [Jakobovits et al., Curr Opin Biotechnol. October; 6(5): 561-6 (1995); Lonberg N. and D. Huszar. Internal Review of Immunology 13: 65-93 (1995); Tomizuka K. et al., Proc. Natl. Acad, Sci. USA 97(2): 722-727 (2000)]. The humanised or human chimeric antibodies of the invention are preferably produced by way of recombinant DNA techniques known to those skilled in the art. The monoclonal antibodies of the invention may preferably be produced by an isolated cell, for example selected from SP2/0, YB2/0, IR983F, Namalwa human myeloma, PERC6, the CHO lines, especially CHO-K-1, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag 14 and P3X63Ag8.653, this list not being limited.

The monoclonal antibodies of the invention may also be produced by way of a transgenic animal. Transgenesis is a molecular genetic technique by which exogenic DNA is introduced into the genome of a multicellular organism and is transmitted to the progeny thereof. This transmission to progeny imposes stable integration of said DNA in the genome of the embryo, at an early stage of development. For example, one of the transgenesis techniques likely to be used within the scope of the invention consists in micro-injecting naked DNA into the pronucleus in a fertilized mammal ovocyte or into embryonic stem cells, which leads, in a certain number of cases, to the integration of part of microinjected DNA molecules into the host genome. Other techniques may be used for transgenesis, and especially techniques for introducing exogenic DNA into a living cell, which are well known to those skilled in the art, especially electroporation, transfection by means of calcium phosphate precipitates, modified liposomes or lipids such as Lipofectamine® (IN VITROGEN).

The monoclonal antibody of the invention is preferably produced by the transgenic animal in its milk. In this way, the gene coding for the protein of interest is associated with gene-regulating elements expressed specifically in milk (for example the promoter of the WAP gene, whey acidic protein). The resulting expression vector is micro-injected under microscope into mammalian embryos at the unicellular stage. The embryos are then transferred to receiving females.

For example, after one month of gestation, the first mammals which had integrated the transgene (F0) into their genome are being born and are identified by ear biopsy PCR analysis. They will be used as founders to give birth to the second generation of transgenic mammals. The founders are selected for their efficiency to produce the protein of interest in their milk and for generating the second generation of transgenic rabbits (F1).

The F1 progeny is identified by ear biopsy followed by PCR analysis. The sexually mature F1 females are then inseminated with sperm from non-transgenic males. The milk is harvested mechanically and the recombinant protein is characterised such as to select the best line for large-scale production and for developing the purification strategy (GLP, pre-GMP, GMP).

In parallel, the sperm of F1 transgenic males—Master Sperm Bank, MSB—is harvested and cryo-conserved in liquid nitrogen, following recommendations of the FDA and of European instances. This sperm will be used for artificially inseminating non-transgenic females to generate the second progeny (F2). The sperm of F2 transgenic males—Working SpermBank, WSB—is harvested and over 15 to 20 years will serve to generate F3 transgenic females which will produce industrial quantities of the monoclonal antibody in their milk. This type of technique is described for example in patent EP 0 527 063.

Fc Gamma Receptors

CD16, also called type III Fc gamma receptor (FcγRIII), is a receptor present on numerous immune system cells. Together with CD32 (FcγRII) and CD64 (FcγRI), CD16 is a specific receptor for constant (Fc) fragments IgG antibodies heavy chains. Binding of an immune complex, via the Fc of IgG, to these CD16, CD32 and CD64 receptor which are present on the immune system effector cells activates the latter and especially immune complex phagocytosis.

The effector cells of the invention express on their cellular membrane 3 types of Fc receptors: CD64, CD32 and CD16. The CD16 receptor is traditionally called “low-affinity receptor”, and is expressed constitutionally on the PMNs (polymorphonuclear neutrophils), a sub-population of monocytes, on macrophages, dendritic cells and Natural Killer cells (NK cells). CD16 participates in multiple effector functions, for example phagocytosis, opsonisation of particles or of immune complexes, and ADCC activity.

The monoclonal antibody of the kit of parts of the invention has an Fc region exhibiting strong affinity for the Fc receptors which are present on the effector cells of the invention, and in particular for CD16. The invention describes the synergy between the Fc region of the monoclonal antibodies of the invention and CD16 of the effector cells of the kit of parts. This affinity is such that the addition of human polyvalent plasmatic IgG (important constituent of peripheral blood) in the medium containing antibodies and effector cells has no or little influence on ADCC activity generated by the association between the monoclonal antibody and the effector cells. This is due to the fact that the affinity of the Fc region of the monoclonal antibody for CD16 is greater than that of the human IgG which are present in physiological conditions. As a consequence, ADCC activity observed in vitro will not be diminished in vivo following the absence of displacement of the antibody of interest by seric IgG. In effect, plasma and serum contain strong concentrations of polyvalent immunoglobulins (also called polyvalent plasmatic IgG or polyclonal IgG or seric IgG). The monoclonal antibody of the kit of parts induces activation of the effector cells via the Fc receptors the CD16 and the CD64 of which lead to cellular lysis by ADCC or phagocytosis. It is now commonly admitted that polyvalent plasmatic IgG inhibit the lysis mechanism of the effector cells via CD64, the latter being saturated in the presence of polyvalent IgG.

The applicant has shown that the association in a kit of parts of a monoclonal antibody the Fc region of which has a greater affinity for CD16 than that of the plasmatic fractions isolated IgG surprisingly induces an ADCC activity which is not inhibited by the addition of plasmatic IgG in vitro, thus making feasible to conserve the therapeutic activity in vivo. This in vivo therapeutic activity corresponds to the lysis of tumour cells, of cells infected by pathogenic agents or of cells producing auto-antibodies. Therefore, advantageously, the monoclonal antibody is not displaced by polyvalent IgG in the case of the addition of human plasmatic IgG.

Due to the strong affinity of the antibody Fc region for CD16, the monoclonal antibody binds to the effector cells, and this binding is not displaced by the human polyvalent plasmatic IgG, even at strong serum concentrations. As a consequence, the kit of parts of the invention enables an optimal lysis of the target cells even at low concentrations of the monoclonal antibody. Advantageously, the concentration of the monoclonal antibody of the kit of parts is less than the concentration of an antibody with the same specificity, traditionally used in monotherapy for treating malignant pathologies, auto-immune or infectious diseases.

In a particular embodiment of the invention, the Fc region of the monoclonal antibody of the invention has an association constant with the CD16 of at least 2.10⁶ M⁻¹. Advantageously, the association constant of the antibody of the invention is measured according to the method described in the document Maenaka et al. (Katsumi Maena, P. Anton van der Merwe, David I. Stuart, E. Yvonne Jones, and Peter Sondermann; The Human Low Affinity Fcy receptors IIa, Iib, and III bind IgG with Fast Kinetics and Distinct Thermodynamic Properties. J. Biol. Chem., Vol. 276, Issue 48, 44898-44904, Nov. 30, 2001).

In a preferred embodiment of the invention, the monoclonal antibody concentration in the kit of parts is preferably less than 1 mg/200 millions of cells. The use of the invention, hereinabove called a kit of parts, is considered in pathologies or after injection. The effector/target ratio is not necessarily high, i.e. less than 10, or even 1 or 0.1.

In a particular aspect of the invention, the monoclonal antibody binds of the effector cell CD16 with an affinity of at least 2.10⁶ M⁻¹. For example, the monoclonal antibody of the invention may be prepared by means of the process described in patent application WO 01/77181. This process for preparating a monoclonal antibody capable of activating the CD16 expressing effector cells comprises the following steps:

a) purificating monoclonal antibodies obtained from various clones originating from cell lines selected from hybridomes, especially heterohybridomes and animal or human cell lines transfected by means of a vector comprising the gene coding for said antibody;

b) adding each antibody obtained in step a) in a distinct reactional mixture comprising:

i. the target cells of said antibody,

ii. effector cells comprising FcγRIII expressing cells

iii. polyvalent IgG,

c) determinating the lysis percentage of target cells and selecting monoclonal antibodies which activate the effector cells causing a significant lysis of the target cells (ADCC activity depending from FcγRIII).

For each antigenic specificity, the monoclonal antibody of the invention is in reality a composition containing monoclonal antibodies, all of them being identical at the level of their primary structure since they all originate from the same cellular clone. However, all antibodies of a monoclonal antibodies composition do not exhibit the same glycannic profile. Human and animal antibodies have a N-bond oligosaccharide on the CH2 domain of each of their heavy chains. The binding site of this oligosaccharide is, for G immunoglobulins, asparagine 297 (Asn 297 according to Kabat). This asparagine residue is also called “Fcγ glycosylation”.

The extremity of the oligosaccharide chain bound to Asn 297 is called “reductor extremity”, whereas the opposite extremity is called “non reductor extremity”. In the Fc region of the IgG antibodies, there are two Fcγ glycosylation sites; therefore two oligosaccharide chains are bound to each antibody molecule.

Therefore, in a monoclonal antibodies composition, the oligosaccharide chains have varied structures, depending from the glycosylation conferred by the productive cell line. However, these chains have a common base structure:

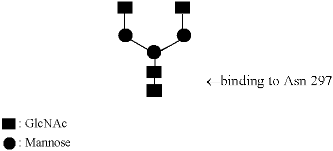

This base structure, common to all monoclonal antibodies, may further comprise the following sugars: N-acetylglucosamine (GIcNAc), fucose (fuc) and galactose (gal). The principal glycosylated forms of N-oligosaccharides are shown below:

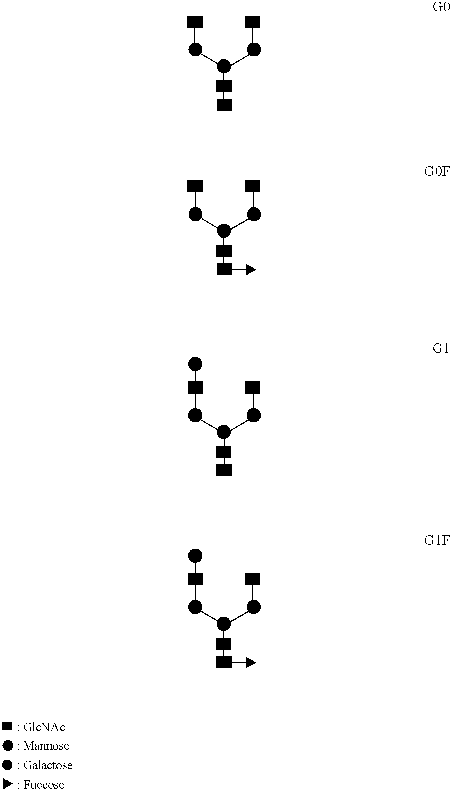

Since each oligosaccharide chain may include one or more of these sugars, and may thus present itself in the herein above illustrated G0, G0F, G1 or G1F form, there is, in a monoclonal antibodies composition, a multitude of combinations of oligosaccharides conferring to the monoclonal antibodies composition a ratio in each of these sugars which may be different from one antibody composition to the other. Therefore, clones originating from the same cell line may produce antibody compositions the glycannic compositions of which may vary. Therefore, it has been shown, surprisingly, by the applicant that the monoclonal antibodies compositions in which the rate of fucose is less than 65% have a strong affinity for CD16. More particularly, this type of monoclonal antibodies composition has an affinity of their region for CD16 which is higher than that of the polyvalent IgG for CD16. In addition, the monoclonal antibodies of the composition are not displaced by seric Ig.

A method for preparating of such monoclonal antibodies compositions is given for example in patent application WO 01/77181. In an advantageous embodiment of the invention, the monoclonal antibodies composition is produced by a cell having low enzymatic activity allowing the addition of fucose to N-acetylglucosamine of the reducing extremity, such an enzyme being preferably fucosyltransferase. In another embodiment of the invention, it is possible to have act on the monoclonal antibodies composition an enzyme, for example fucosidase, so as to obtain a monoclonal antibodies composition comprising such a rate of fucose. In a preferred embodiment of the invention, the monoclonal antibodies composition is produced in YB2/0 (ATCC CRL-1662).

By way of advantage, the monoclonal antibody of the kit of parts of the invention is directed against the 5C5 antigen (tumorous antigen expressed by the cells of renal carcinomas), BCR (B Cell Receptor), an idiotype such as that of anti-FVIII inhibitors antibodies, TCR (T Cell Receptor), CD2, CD3, CD4, CD8, CD14, CD15, CD19, CD20, CD21, CD22, CD23, CD25, CD45, CD30, CD33, CD37, CD38, CD40, CD40L, CD46, CD52, CD54, CD66 (a,b,c,d), CD74, CD80, CD86, CD126, CD138, CD154, MUC1 (Mucine 1), MUC2 (Mucine 2), MUC3 (Mucine 3), MUC4 (Mucine 4), MUC16 (Mucine 16), HM1.24 (specific antigen for plasmocytes which is overexpressed in multiple myelomas), tenascin (protein of the extra-cellular matrix), GGT (gamma-glutamyltranspeptidase), VEGF (Vascular Endothelial Growth Factor), EGFR (Endothelial Growth Factor receptor), CEA (carcinoembryonic antigen), CSAp (colon-specific antigen-p), ILGF (Insulin-Like Growth factor), placental growth factor, Her2/neu, carbonic anhydrase IX, IL-6, S100 proteins (multigenic family of proteins linking to calcium), MART-1 (tumorous differentiation antigen associated with melanoma), TRP-1 (tyrosinase-related protein 1), TRP-2 (tyrosinase-related protein 2), gp100 (glycoprotein 100 kDa), amyloid proteins, rhesus D antigen, MHC molecules of class I and II such as HLA-DR, an antigen resulting from the expression of mutated genes especially oncogenes or tumour-suppress genes, an antigen derived from oncogenic viruses which have expressed by certain tumours, a ubiquitous antigen overexpressed in some tumours and slightly expressed in some normal tissues, such as for example the type II receptor of the Müllerian hormone, a glycosylated or non-glycosylated protein, a phospholipid, a molecule of the self or of the non-self expressed or exposed on the membrane by infected cells such as phosphatidylserine, and a protein expressed or secreted by a pathogenic agent (bacterial toxin, protein complexes of the bacterial or parasitic wall, viral envelope glycoproteins, for example from HIV virus, HBV, HCV, RSV, etc.), this list not being limited.

The antibody of the invention is preferably directed against the CD20. The CD20 antigen is a hydrophobic transmembrane protein with a molecular weight of 35-37 kDa which is present on the surface of mature B lymphocytes (Valentine et al. 1987, Proc Natl Acad Sci U.S.A. 84(22): 8085-9; Valentine et al. 1989, J. Biol. Chem. 264(19): 11282-11287). It is expressed during the development of B lymphocytes from the early pre-B stage until differentiation in plasmocyte, a stage where this expression disappears. The CD20 antigen is present both on normal B lymphocytes and on malignant B cells. More particularly, the CD20 antigen is expressed on the most of B phenotype lymphomas (80% of lymphomas): it is expressed for example on more than 90% of lymphocytes B non-Hodgkin's lymphomas (NHL), and on more than 95% of B type Chronic Lymphoid Leukemias (LLC-B). The CD20 antigen is not expressed on the haematopoietic stem cells or on the plasmocytes.

The function of CD20 is not yet fully clarified, though it could act as a calcic channel and intervene in the regulation of the first steps of differentiation (Golay et al. 1985, J. Immunol.; 135(6): 3795-801) and of proliferation (Tedder et al. 1986, Eur J. Immunol. 1986 August; 16(8): 881-7) of B lymphocytes. In a preferred embodiment of the invention, the composition of anti-CD20 antibodies is produced by YB2/0 and has a fucose rate of less than 65%. In a particular embodiment of the invention, such an antibody, and its production process, are described in patent application WO2006/064121.

Advantageously, the amino acid sequence of the heavy chain of such an antibody is the sequence set forth in SEQ ID NO: 1. Advantageously, the aminoacid sequence of the light-chain of such an antibody is the sequence set forth in SEQ ID NO: 2 or 3.

In brief, this antibody may be obtained, in accordance with the teaching of patent application WO2006/064121, by means of YB2/0 cell transfection by vectors allowing the expression of the hereinabove described light chain and heavy chain. In a preferred embodiment of the invention, the composition of monoclonal anti-CD20 antibody has a fucose rate of less than 65%, and comprised preferably between 20 and 40%, or a fucose/galactose ratio of less than 0.6. In a preferred embodiment of the invention, the monoclonal antibody of the kit of parts is produced by the R509 clone, deposited to the CNCM under accession number CNCM I-3314. In another preferred embodiment of the invention, the monoclonal antibody of the kit of parts is produced by the R603 clone, deposited to the CNCM under accession number CNCM I-3529.

The applicant has shown that the kit of parts of the invention is efficient for treating LLC-B, since malignant cells from patients with LLC-B were lysed ex vivo, and even at a ratio less than or equal to 10, or even 5 or even 2 E:T, at low antibody concentrations, including in the presence of human serum. The kit of parts of the invention thus allows optimal lysis of the target recognised by the variable regions of the antibody, due to the physical interactions (binding) between the effector cells and the Fc region of the antibodies, which is sufficiently strong not to be displaced by the polyvalent IgG. Advantageously, the concentration of monoclonal antibody contained in the kit of parts of the invention for treating LLC-B is of less than 375 mg/m².

Because of its advantages as regards of low toxicity, specificity and reduced dose, the kit of parts comprising the anti-CD20 antibodies may be administered for treating the following pathologies: malignant pathologies with a lymphoproliferative syndrome of CD20 positive B lymphocytes with for example type B NHL or acute or chronic lymphoid B leukemias, auto-immune and/or inflammatory diseases such as organ grafts rejection, graft versus host disease, rheumatoid polyarthritis, disseminated lupus erythematosus, sclerodermia, primitive Sjögren's syndrome (or Gougerot-Sjögren's Syndrome), auto-immune polyneuropathies such as multiple sclerosis, type I diabetes, auto-immune hepatitis, ankylosing spondylarthritis, Reiter's syndrome, gout arthritis, coeliac disease, Crohn's disease, Hashimoto's thyroiditis, Addison's disease, auto-immune hepatitis, Basedow's disease, ulcerative colitis, vasculitis such as systemic vasculitis associated with ANCA (anti-neutrophil cytoplasmic antibody), auto-immune cytopenias and other haematological complications in adults and children, such as acute or chronic auto-immune thrombopenias, auto-immune haemolytic anaemias, haemolytic disease of the newborn (HDN), cold agglutinin disease, thrombocytopenic thrombotic purpura and acquired auto-immune haemophilia; Goodpasture's syndrome, extra-membraneous nephropathies, auto-immune bullous skin disorders, refractory myasthenia, mixed cryoglobulinemias, psoriasis, juvenile chronic arthritis, inflammatory myositis, dermatomyositis and children systemic auto-immune diseases including antiphospholipids syndrome, this list not being limited.

Advantageously, the kit of parts of the invention is an injectable solution. This injectable solution is advantageously in the form of a locally or systemically injectable solution. In a particular embodiment, 6 administrations are done to the patient. One administration is done per day or every two days over a week, then once per week over one month or two, one administration three times/month, the cure being renewable several times.

In a complementary embodiment, the effector cells are administered at a dose comprised between 10⁴ and 10⁹ effector cells per injection. In another complementary embodiment, the antibodies of the invention are administered at a dose comprised between 1 and 500 mg of antibodies per injection.

In another particular embodiment of the invention, the effector cells are administered repeatedly up to 10 times, the time interval between each administration being comprised between 2 days and 12 months. In another particular embodiment of the invention, the monoclonal antibody is administered repeatedly up to 10 times, the time interval between each administration being comprised between 2 days and 12 months. In another embodiment of the invention, the monoclonal antibody and the effector cells are administered simultaneously.

In another embodiment of the invention, the monoclonal antibody and the effector cells are administered sequentially, the monoclonal antibody being administered before the effector cells. In another embodiment of the invention, the monoclonal antibody and the effector cells are administered sequentially, the monoclonal antibody being administered after the effector cells. Another object of the invention is a pharmaceutical composition comprising the kit of parts of the invention. Another object of the invention relates to the use of the kit of parts of the invention for preparing a drug for treating malignant, auto-immune and infectious pathologies.

This drug or pharmaceutical composition advantageously comprises an excipient and/or a pharmaceutically acceptable vehicle. The excipient may be any solution, such as a saline, physiological, isotonic, buffered solution, etc., as well as any suspension, gel, powder, etc., compatible with pharmaceutical usage and known to those skilled in the art. The compositions according to the invention may also contain one or more agents or vehicles selected from dispersants, solubilisers, stabilisers, surfactants, preservatives, etc. Also, the compositions according to the invention may comprise other agents or active ingredients.

Another object of the invention is the use of the kit of parts of the invention for manufacturing a drug. Another object of the invention is the use of the kit of parts of the invention for manufacturing a drug for treating a malignant pathology.

Advantageously, this malignant pathology is selected from solid tumours and malignant haemopathies. Solid tumours are selected from melanomas, carcinomas, sarcomas, gliomas and skin cancers. Carcinomas are selected in the group constituted by kidneys, breast, oral cavity, lungs, gastro-intestinal tract, ovaries, prostate, uterus, bladder, pancreas, liver, gallbladder, skin and testicles carcinomas. Malignant haemopathies are selected from lymphoproliferative, myeloproliferative, myelodysplasic syndromes and acute myeloid leukemias with for example type B NHL, acute or chronics lymphoid B leukemias, Burkitt's lymphoma, tricholeucocyte leukaemia, acute and chronic myeloid leukemias, T lymphomas and leukemias, Hodgkin's lymphomas, Waldenström's macroglobulinemia and multiple myelomas, this list not being limited.

Another object of the invention is the use of the kit of parts of the invention for manufacturing a drug intended for treating an auto-immune and/or inflammatory primitive or secondary condition, which is specific to organs or systemics and which is associated or not with pathogenic auto-antibodies, selected from organ grafts rejection, graft versus host disease, rheumatoid polyarthritis, disseminated lupus erythematosus, sclerodermia, primitive Sjögren's syndrome (or Gougerot-Sjögren syndrome), auto-immune polyneuropathies such as multiple sclerosis, type I diabetes, auto-immune hepatitis, ankylosing spondylarthritis, Reiter's syndrome, gout arthritis, coeliac disease, Crohn's disease, Hashimoto's thyroiditis, Addison's disease, auto-immune hepatitis, Basedow's disease, ulcerative colitis, vasculitis such as systemic vasculitis associated with ANCA (antineutrophil cytoplasmic antibody), auto-immune cytopenias and other haematological complications in adults and children, such as acute or chronic auto-immune thrombopenias, auto-immune haemolytic anaemias, haemolytic disease of the newborn (HDN), cold agglutinin disease, thrombocytopenic thrombotic purpura and acquired auto-immune haemophilia; Goodpasture's syndrome, extra-membraneous nephropathies, auto-immune bullous skin disorders, refractory myasthenia, mixed cryoglobulinemias, psoriasis, juvenile chronic arthritis, inflammatory myositis, dermatomyositis and children systemic auto-immune diseases including antiphospholipids syndrome, this list not being limited.

Another object of the invention is the use of the kit of parts of the invention for manufacturing a drug for treating an infectious disease. Advantageously, this infectious disease is selected from those induced by virus (human immunodeficiency virus or HIV, hepatitis B or C virus (HBV, HCV), Epstein-Barr virus or EBV, cytomegalovirus or CMV, enterovirus, influenza with the A, B and C Influenza virus, respiratory syncytial virus or RSV, or HTLV), bacteria and/or their toxins (tetanus, diphtheria, pneumococci, meningococci, staphylococci including methicilin resistant forms, Klebsiellas, Shigellas, pseudomonas aeruginosa, enterobacteria or antibiotics resistant pathologies including nosocomial diseases), parasites (paludism, leishmaniosis, trypanosomiases) as well as emerging diseases, for example Chikungunya, bird flu, severe acute respiratory virus syndrome or SARS, viruses responsible for haemorrhagic fevers such as Ebola or Dengue fever or West Nile virus, and those related to bio-terrorism, such as Anthrax, Botulism, Plague, smallpox and poxvirus, Tularaemia, haemorrhagic fever agents, brucellosis, Staphylococcus B Enterotoxins, diphtheric toxin or viral Encephalitis, this list not being limited. Other aspects and advantages of the invention will be described in the following examples which must be considered as illustrative and do not limit the scope of the invention.

### BRIEF DESCRIPTION OF DRAWINGS

FIG. 1: Binding study of anti-D R297 EMABling antibodies and of AD1 antibody to CD16 (FcγRIII receptor) of macrophages through a competition test;

FIG. 2: Macrophage induced ADCC activity of EMABling R297 antibodies and of AD1 antibody in the presence of various concentrations of polyclonal immunoglobulins (IVIg);

FIG. 3: Macrophage induced ADCC activity of EMABling R297 antibodies and of AD1 antibody in the presence of various concentrations of immunoglobulins (IVIg) and of anti-CD16 3G8 antibody at a concentration of 6.25 μg/ml; and

FIG. 4: Phagocytosis of Rh+erytrhocytes by CD16+ macrophages induced by the EMABling R297 antibody and the AD1 antibody in the presence of various concentrations of immunoglobulins (IVIg).

### EXAMPLES

### Example 1

### Differentiation of Monocytes in Macrophages

Monocytes are isolated from peripheral blood by fractionating on Ficol and Percol density gradient, then culturing in an RPMI medium containing 10% SVF and adding of M-CSF (Monocyte Colony Stimulating Factors) (50 ng/ml). After 7 days, the obtained macrophages are of CD14+, CD16+, CD32+, CD64+, CD11b+, CD1a−, CD80−, CD83− phenotype. Therefore, the M-CSF differentiation allows expression of CD16 on the surface of macrophages.

### Example 2

### Interaction of Anti-D Antibodies with CD16 Expressed by Macrophages

The binding of the anti-D R297 antibody (also called “EMABling R297”) is compared to that of the AD1 antibody. The anti-D R297 antibody is described in the document WO 01/77181, and is produced according to the process described in this document. This antibody is produced in the YB2/0 cell (ATCC CRL-1662). Binding of the R297 antibody on macrophages CD16 is compared to that of the AD1 antibody (described in the document WO 01/77181, expressed by a heteromyeloma).

The displacement assay of the anti-CD16 antibody (producer clone 3G8) allows to measure the binding of monoclonal antibodies on the CD16 receptor of these macrophages, irrespective of their specificity. The purified macrophages are incubated with variable concentrations (0 to 83 μg/ml) of anti-D antibody (R297 or AD1) and with the anti-CD16 3G8 antibody coupled to a fluorochrome (3G8-PE) at a determined concentration.

After washing, binding of the antibody 3G8-PE on the CD16 receptor of the macrophages is evaluated by flow cytometry. The antibodies having the capacity to bind themselves on CD16 enter into competition with the binding of the 3G8 antibody and, consequently, induce a decrease in MFI (Mean Fluorescence Intensity). The results are expressed in fluorescence averages (MFI), as a function of the quantity of antibodies to be evaluated.

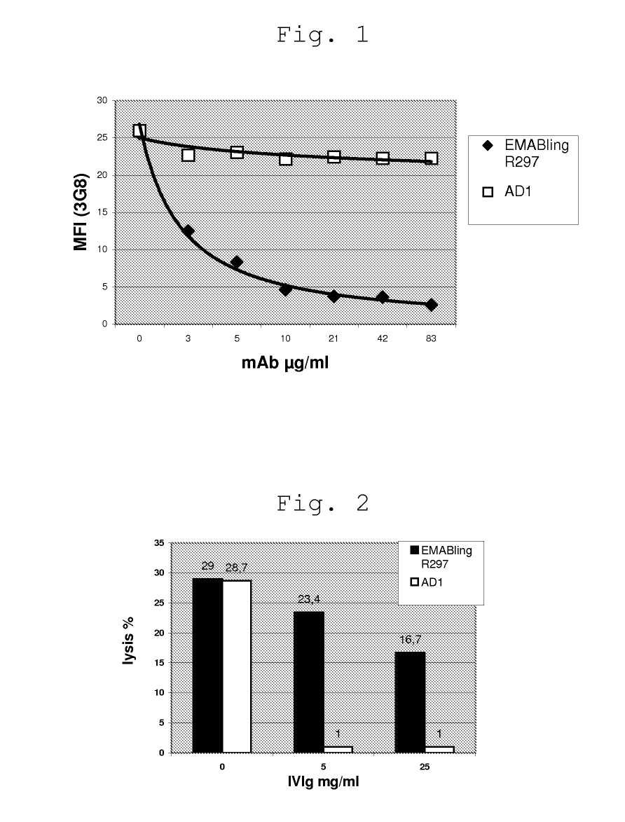

FIG. 1 shows that the R297 antibody binds very strongly on macrophages CD16 when compared to the AD1 antibody. At the plateau, the EMABling antibody induces a displacement which is at least 6 times greater than the AD1 antibody.

### Example 3

### Interaction of Anti-CD20 EMAB6 and EMAB603 Antibodies with CD16 Expressed by Macrophages

The binding of anti-CD20 EMAB603 antibodies (produced by the R603 clone, deposite to the CNCM under the number I-3529) and EMAB6 (produced by the R509 clone, deposited to the CNCM under the number I-3314) on macrophages CD16 is compared to that of Rituxan. The anti-CD20 EMAB6 and EMAB603 antibodies are produced according to the process described in patent application WO2006/064121, in particular at pages 26-33. Also, the clones producing these antibodies are available at CNCM under the accession numbers CNCM I-3314 and CNCM I-3529, respectively. The displacement assay of the anti-CD16 antibody (producer clone 3G8) measures the binding of the monoclonal antibodies on the CD16 receptor, irrespective of their specificity.

The macrophages are incubated with variable concentrations (0 to 83 μg/ml) of anti-CD20 antibody (EMAB6, EMAB603 or rituximab) and with the anti-CD16 3G8 antibody coupled to a fluorochrome (3G8-PE) at a determined concentration. After washing, binding of the 3G8-PE antibody on the CD16 receptor of the macrophages is evaluated by flow cytometry. The antibodies having the capacity to bind themselves on CD16 enter into competition with the binding of the 3G8 antibody, and consequently induce a decrease in MFI (Mean Fluorescence Intensity). The results are expressed in fluorescence averages (MFI), as a function of the quantity of antibodies to be evaluated.

The Imax values (maximal inhibition of 3G8 binding) and IC50 values (anti-CD20 antibody concentration required to induce a 3G8 binding inhibition of 50% of Imax) are calculated using PRISM statistical analysis software.

Result: the interaction of EMABling R603 and EMAB6 antibodies on macrophages CD16 is much greater than that obtained with Rituxan. Therefore, since this assay is performed in the absence of an antigenic target, the anti-CD20 antibodies of the invention have the capacity to be bind strongly on macrophages CD16.

### Example 4

### Anti-D/Erytrhocytes Rh+/Macrophages ADCC Activity. Role of IVIg Polyvalent (Tegeline®)

The cytotoxic capacity of anti-D antibodies is studied by the ADCC technique. The anti-D antibodies, macrophages (differentiated monocytes in M-CSF) and Rhesus D+ erythrocytes (effector/target ratio of around 2/1) are incubated for 16 h at 37° C. in the presence of various concentrations of polyvalent immunoglobulins (IVIg) (Tegeline®). The cytotoxic activity induced by the antibodies is then measured by colorimetry in quantifying in the supernatants the haemoglobin released by the lysed erythrocytes. The results of specific lysis are expressed in lysis percentage.

The results of FIG. 2 indicate that in the presence of macrophages, the EMABling R297 antibody has a strong remaining ADCC activity in the presence of significant concentrations of IVIg, contrary to the AD1 antibody which solely induces lysis by ADCC in the absence of IVIg. Therefore, in the absence of polyvalent immunoglobulins, the two anti-D antibodies, EMABling R297 and AD1 have an ADCC activity of the order of 29%. On the contrary, at the concentration of 5 mg/ml of polyvalent immunoglobulins, the EMABling antibody appears at least 20 times more active (23% lysis versus 1% with AD1). This advantage subsists at stronger concentrations of polyvalent immunoglobulins (25 mg/ml), the respective percentages of lysis for the EMABling and AD1 antibodies being 16 and 1%.

### Example 5

### Anti-D/Erythrocytes Rh+/Macrophages ADCC Activity. Role of Polyvalent IVIg (Tegeline®)

According to the same protocol as described in example 4, the ADCC activity of anti-CD20 was also studied in the presence of Raji cells and macrophages (differentiated monocytes in M-CSF). The anti-CD20 antibodies (produced by the R603 clone or Rituxan) are incubated in the presence of macrophages, Raji cells and various concentrations of polyvalent IVIg (Tegeline®). After 16 h of incubation at 37° C., the ADCC activity induced by the antibodies is measured by colorimetry in quantifying in the supernatants the quantity of intracellular LDH (lactate deshydrogenase) released by the Raji cells. The results of specific lysis are expressed in lysis percentage.

The results indicate that the anti-CD20 R603 antibody has an ADCC activity of at least 2 times greater than that induced by Rituxan in the presence of macrophages expressing CD16 and Tegeline®. This ADCC activity depends from CD16 expressed by the macrophages such as shown by the inhibitor effect of the anti-CD16 3G8.

### Example 6

### Anti-D/Etythrocytes Rh+/Macrophages ADCC Activity. CD16 Highlighting in the Presence of IVIg

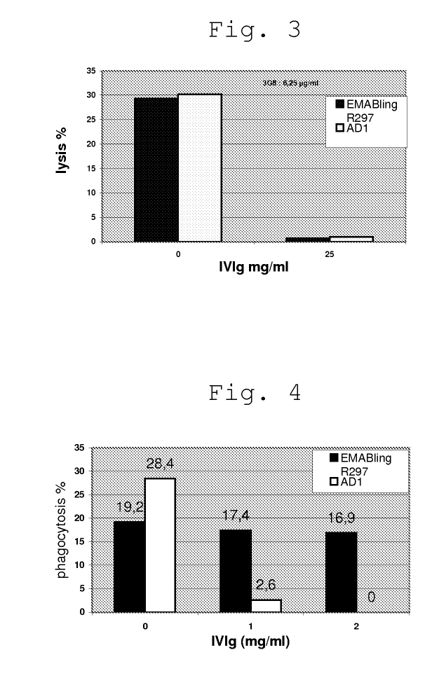

The addition of anti-CD16 antibody, 3G8, inhibits the ADCC induced by the EMABling antibody in the presence of the strongest concentration of IVIg tested, indicating that the induced lysis depends from the CD16 expressed on the macrophages (FIG. 3).

### Example 7

### Phagocytosis of Rhesus+Erythrocytes by CD16+ Macrophages Induced by the EMABling R297 Antibody in the Presence of IVIg

The capacity of anti-D R297 antibodies to induce phagocytosis of Rhesus+erythrocytes by CD16+ macrophages is studied by flow cytometry. The anti-D antibodies, the macrophages labelled with PKH67 (differentiated monocytes M-CSF and Rhesus D+ erythrocytes (effector/target ratio of 5/1) labelled with PKH26 are incubated for 3 h at 4° C. and 37° C. in the presence of various concentrations of polyvalent IVIg (Tegeline®).

The results correspond to the percentage of PKH67/PKH26 doubly labelled, i.e. having phagocyted at least one erythrocyte.

Results: at 4° C., the macrophages and erythrocytes appear in different windows in cytometry, each being labelled with a specific fluorochrome. The phagocytosis percentage is very low, of the order of 4% in the absence of IVIg, and from 1 to 2% in the presence of IVIg. These values at 4° C. are systematically deduced to formulate the phagocytosis percentage at 37° C.

At 37° C., the percentage of PKH67/PKH26 doubly labelled increases in the absence of IVIg for the two assayed antibodies, R297 EMABling and AD1. In the presence of IVIg, only the EMABling antibody has the capacity to phagocyte the Rh+erythrocytes, contrary to the AD1 antibody. Therefore, should there be 0, 1 or 2 mg/ml of IVIg, the percentage of phagocytosis remains between 15 and 20%, showing that the addition of IVIg does not inhibit phagocytosis induced by the EMABling antibody.

At a concentration of 1 mg/ml, the EMABling antibody is at least 5 times greater than the AD1 antibody. At a concentration higher than 2 mg/ml, the phagocytosis percentage is of 16.9% with the EMABling antibody and not significant (value 0) with the AD1 antibody.

### Example 8

### Phagocytosis of Rhesus+Erythrocytes by CD16+ Macrophages Induced by the R603 Antibody in the Presence of IVIg

Phagocytosis of CD20 Raji cells in the presence of CD16 macrophages, induced by the R603 antibody in the presence of IVIg was also studied. The capacity of anti-CD20 antibodies to induce phagocytosis of Raji cells by CD16+ macrophages is studied by flow cytometry. Anti-CD20 antibodies, PKH67 labelled macrophages (differentiated monocytes M-CSF) and the Raji cells (effector/target ratio of 5/1, 10/1 and 20/1) labelled with PKH26 are incubated for 3 h at 4° C. and 37° C. in the presence of various concentrations of polyvalent IVIg (Tegeline®).

The results correspond to the percentage of PKH67/PKH26 doubly labelled, having phagocyted at least one Raji cell.

Results: At 4° C., the macrophages and Raji cells appear in different windows in cytometry, each being labelled by a specific fluorochrome. The percentage of phagocytosis is very low, less than 5% in the absence and in the presence of IVIg. These values at 4° C. are systematically deduced to formulate the phagocytosis percentage at 37° C. At 37° C., the percentage of PKH67/PKH26 doubly labelled increases in the absence of IVIg for the two antibodies tested, anti-CD20 R603 and Rituxan.

In the presence of IVIg, the EMABling antibody has a greater capacity, of the order of 2 times, 4 times, or even 10 times for phagocyting the Raji cells when compared to the Rituxan antibody. Therefore, should there be 0; 1 or 2 mg/ml of IVIg, the percentage of phagocytosis always remains greater than that induced by Rituxan, showing that in the presence of IVIg the EMABling antibody induces phagocytosis in the presence of CD16+ Macrophages.

1. A kit of parts for treating a malignant pathology, an auto-immune disease or an infectious disease, comprising an effector cell which expresses the FcγRIII receptor (CD16) on its surface, and a monoclonal antibody, in which the affinity of the Fc region of said monoclonal antibody for CD16 is greater than the affinity of the Fc region of the polyclonal immunoglobulins for CD16.

2. The kit of parts according to claim 1, wherein said effector cell which expresses the FcγRIII receptor (CD16) on its surface is a monocyte or a monocyte or monocyte precursor derived cell which expresses the FcγRIII receptor (CD16) on its surface.

3. The kit of parts according to claim 1, wherein said monocyte or monocyte precursor derived cell which expresses CD16 on its surface is selected from monocytes expressing CD16, macrophages, Natural Killer cells (NK), dendritic cells and all Peripheral Blood Mononuclear Cell (or PBMC).

4. The kit of parts according to claim 3, wherein said monocyte or monocyte precursor derived cell, which expresses CD16 on its surface is a macrophage.

5. The kit of parts according to claim 1, wherein said monoclonal antibody is not displaced by polyclonal immunoglobulins, particularly those present in human serum, due to said affinity of the Fc region of said monoclonal antibody for CD16.

6. The kit of parts according to claim 1, wherein said monoclonal antibody binds CD16 of said monocyte or monocyte precursor derived cell with an affinity greater than 2.10⁶ M⁻¹.

7. The kit of parts according to claim 1, wherein said monoclonal antibody is produced in the form of a monoclonal antibodies composition, wherein each antibody has N-linked sugar chains linked at the Fcγ glycosylation site (asparagine 297, according to Kabat), and wherein among all the N-linked sugar chains at said glycosylation site of all the antibodies of said composition, the rate of fucose is less than 65%.

8. The kit of parts according to claim 1, wherein said monoclonal antibody is directed against an antigen selected from the 5C5 antigen (tumorous antigen expressed by the cells of renal carcinomas), BCR (B Cell Receptor), an idiotype such as that of anti-FVIII inhibitor antibodies, TCR (T Cell Receptor), CD2, CD3, CD4, CD8, CD14, CD15, CD19, CD20, CD21, CD22, CD23, CD25, CD45, CD30, CD33, CD37, CD38, CD40, CD40L, CD46, CD52, CD54, CD66 (a, b, c, d), CD74, CD80, CD86, CD126, CD138, CD154, MUC1 (Mucine 1), MUC2 (Mucine 2), MUC3 (Mucine 3), MUC4 (Mucine 4), MUC16 (Mucine 16), HM1.24 (specific antigen for plasmocytes which is overexpressed in multiple myelomas), tenascin (protein of the extra-cellular matrix), GGT (gamma-glutamyltranspeptidase), VEGF (Vascular Endothelial Growth Factor), EGFR (Endothelial Growth Factor receptor), CEA (carcinoembryonic antigen), CSAp (colon-specific antigen-p), ILGF (Insulin-Like Growth factor), placental growth factor, Her2/neu, carbonic anhydrase IX, IL-6, S100 proteins (multigenic family of proteins binding to calcium), MART-1 (tumorous differentiation antigen associated with melanoma), TRP-1 (tyrosinase-related protein 1), TRP-2 (tyrosinase-related protein 2), gp100 (glycoprotein 100 kDa), amyloid proteins, rhesus D antigen, MHC molecules of class I and II such as HLA-DR), an antigen resulting from the expression of mutated genes, especially oncogenes or tumour-suppressor genes, an antigen derived from oncogenic viruses expressed by certain well defined tumours, an ubiquitous antigen overexpressed in some tumours and slightly expressed in some normal tissues such as for example the type II receptor of the Müllerian hormone, a glycosylated or non-glycosylated protein, a phospholipid, a molecule of the self or of the non-self expressed or exposed at the membrane by infected cells such as phosphatidylserine, and a protein expressed or secreted by a pathogenic agent (bacterial toxin, proteins complexes of the bacterial or parasitic wall, viral envelope glycoproteins, for example of HIV virus, HBV, HCV and RSV).

9. The kit of parts according to claim 8, wherein said monoclonal antibody is directed against CD20.

10. The kit of parts according to claim 9, wherein said anti-CD20 antibody is produced by the cell line R509 deposited to the CNCM under the accession number I-3314, or by the cell line R603, deposited to the CNCM under the accession number I-3529.

11. The kit of parts according to claim 1, for use in therapy, simultaneously, sequentially or separately.

12. The kit of parts according to claim 1, wherein said effector cell expressing CD16 on its surface has a cytotoxic activity over the target cell of said antibody which is favoured with the interaction of the antibody with CD16.

13. The kit of parts according to claim 1, wherein said monoclonal antibody induces cytotoxicity by ADCC activity or by phagocytosis of said target cell of the antibody in the presence of an effector cell expressing CD16.

14. A pharmaceutical composition containing a kit of parts according to claim 1, and pharmaceutically acceptable excipients.

15. A use of a kit of parts according to claim 1, for manufacturing a drug.

16. A use of a kit of parts according to claim 1, for manufacturing a drug for treating a malignant pathology.

17. The use of a kit of parts according to claim 16, for treating a malignant pathology selected from solid tumours and malignant haemopathies.

18. The use of a kit of parts according to claim 17, wherein the solid tumours are selected from melanomas, carcinomas, sarcomas, gliomes and skin cancers.

19. The use of a kit of parts according to claim 18, wherein the carcinomas are selected in the group constituted by kidney, breast, oral cavity, lungs, gastro-intestinal tract, ovaries, prostate, uterus, bladder, pancreas, liver, gallbladder, skin and testicles carcinomas.

20. The use of a kit of parts according to claim 17, wherein the malignant haemopathies are selected from the lymphoproliferative, myeloproliferative, myelodysplasic syndromes and acute myeloid leukemias with type B NHL, acute or chronic B lymphoid leukemias, Burkitt's lymphoma, tricholeucocyte leukaemia, acute and chronic myeloid leukemias, T lymphomas and leukemias, Hodgkin's lymphomas, Waldenström's macroglobulinemia and multiple myelomas.

21. A use of a kit of parts according to claim 1, for manufacturing a drug intended for treating an auto-immune disease and/or primitive or secondary inflammatory disease, which is specific for organs or systemic and which is associated or not with pathogenic auto-antibodies.

22. The use of a kit of parts according to claim 21, for treating an auto-immune disease and/or a primitive or secondary inflammatory disease, which is specific for organs or systemic and which is associated or not with pathogenic auto-antibodies, selected from the organ grafts rejection, the graft versus host disease, rheumatoid polyarthritis, disseminated lupus erythematosus, sclerodermia, primitive Sjögren's syndrome (or Gougerot-Sjögren syndrome), auto-immune polyneuropathies such as multiple sclerosis, type I diabetes, auto-immune hepatitis, ankylosing spondylarthritis, Reiter's syndrome, gout arthritis, coeliac disease, Crohn's disease, Hashimoto's thyroiditis, Addison's disease, auto-immune hepatitis, Basedow's disease, ulcerative colitis, vasculitis such as systemic vasculitis associated with ANCA (Antineutrophil cytoplasmic antibody), auto-immune cytopenias and other haematological complications in adults and children, such as acute or chronic auto-immune thrombopenias, auto-immune haemolytic anaemias, haemolytic disease of the newborn (HDN), cold agglutinin disease, thrombocytopenic thrombotic purpura and acquired auto-immune haemophilia; Goodpasture's syndrome, extra-membraneous nephropathies, auto-immune bullous skin disorders, refractory myasthenia, mixed cryoglobulinemias, psoriasis, juvenile chronic arthritis, inflammatory myositis, dermatomyositis and auto-immune systemic diseases in children including antiphospholipids syndrome.

23. A use of a kit of parts according to claim 1, for manufacturing a drug for treating an infectious disease.

24. The use of a kit of parts according to claim 23, for treating an infectious disease selected from those induced by virus (human immunodeficiency virus or HIV, virus of hepatitis B or C (HBV, HCV), Epstein-Barr virus or EBV, cytomegalovirus or CMV, enterovirus, influenza with Influenza virus A, B and C, syncytial respiratory virus or SRV, or HTLV), bacteria and/or their toxins (tetanus, diphtheria, pneumococci, meningococci, staphylococci including methicilin resistant forms, Klebsiellas, Shigellas, pseudomonas aeruginosa, enterobacteria or antibiotic resistant pathologies including nosocomial diseases), parasites (paludism, leishmaniosis, trypanosomiasis) as well as emerging diseases, for example Chikungunya, bird flu, severe acute respiratory virus syndrome or SARS, virus responsible for haemorrhagic fevers such as Ebola or Dengue fever or west Nile virus, and those related to bio-terrorism, such as Anthrax, Botulism, Plague, smallpox and poxvirus, Tularaemia, haemorrhagic fever agents, brucellosis, B Enterotoxins of Staphylococcus, diphtheric toxin or viral Encephalitis.


SEQUENCE LISTING (3 sequences)

SEQ ID NO: 1 - protein, 448 aa, Artificial Sequence
  Synthetic Construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Synthetic Construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 213 aa, Artificial Sequence
  Synthetic Construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC